(19) United States
(12) Patent Application Publication
(10) Pub. No.: US 2025/0336495 A1
(43) Pub. Date: Oct. 30, 2025

(54) SYSTEM FOR VERIFYING CHEMOTHERAPY MEDICATION PREPARATION AND METHOD THEREOF

(71) Applicants: Southern Taiwan University of Science and Technology, Tainan (TW); NATIONAL DEFENSE MEDICAL CENTER, Taipei (TW)

(72) Inventors: Chiu-Lan YAN, Taipei (TW); I-Hsun LI, Taipei (TW); Ming-Che CHEN, Tainan (TW); Cian-Yan JIAN, Tainan (TW); Min-Yu LIN, Tainan (TW); Shang-Cian WU, Tainan (TW)

(21) Appl. No.: 18/941,729

(22) Filed: Nov. 8, 2024

(30) Foreign Application Priority Data
Apr. 24, 2024 (TW) 113115296

(51) Int. Cl. G16H 20/10; G06K 7/14; G06V 10/74; G06V 40/20

(52) U.S. Cl. CPC G16H 20/10; G06V 10/74; G06V 40/20; G06K 7/1417; G06V 2201/03

(57) ABSTRACT

A system for verifying chemotherapy medication preparation is disclosed, wherein the system includes: an image sensing module for capturing image information of a chemotherapy medication preparation process; an image recognition module that is in signal connection with the image sensing module and is configured to perform image recognition based on the image information and produce the corresponding identification result; and a computation and processing module that is in signal connection with the image recognition module and is configure to perform computation and comparison based on the identification result; to produce a verification result; to output feedback information when the verification result indicates a mismatch, in order for the image information to be recaptured and re-subjected to image recognition until the verification result indicates a match; and to output preparation process information when the verification result indicates a match.

### BACKGROUND OF THE INVENTION

### 1. Cross Reference

This non-provisional application claims priority of Taiwan Invention patent application No. 113115296, filed on Apr. 24, 2024, the contents thereof are incorporated by reference herein.

### 2. Technical Field

The present invention relates to a verification system and a method thereof. More particularly, the invention relates to a system for verifying chemotherapy medication preparation and a method thereof.

### 3. Description of Related Art

According to the statistical data released by the Ministry of Health and Welfare regarding the ten major causes of death in Taiwan in 2022, cancer has topped the list of the ten major causes for 41 consecutive years, accounting for 24.9% of the total number of deaths in 2022, a 1.2% increase from 2021. Also known as malignant tumor, cancer is caused by the activation of an oncogene. The activation results in uncontrolled cell proliferation and consequently the formation of a tumor. People nowadays lead a busy life, generally have bad eating habits and living habits, stay up late frequently, have irregular daily routines, and lack physical exercise. All of these unwholesome ways of living are factors contributing to the risk of cancer development.

Chemotherapy uses chemically synthesized drugs to treat diseases and is currently a major treatment for malignant tumors. Common chemotherapy medication includes such drugs as Adriamycin, cisplatin, cyclophosphamide, and 5-FU. These drugs are cytotoxic and therefore may be harmful to people involved in their preparation or to the environment if not properly handled during the preparation process. Even in a safe operation environment, a pharmacist still has to consider the stability and compatibility of such drugs. A proper choice of solvent, temperature control, and the storage environment play an important role in the safety of chemotherapy drugs.

Given the above, an environment where a chemotherapy medication is prepared should be as far away from other working areas as possible, and unauthorized people should be kept from entering such a medication preparation site when a preparation process is underway. Besides, all medication preparation operations must be performed by those who have received special training so as to reduce the danger of contamination, and during a chemotherapy medication preparation process, the preparation personnel must be familiar with all the preparation steps, including, for example, verifying whether or not the drug label matches the drug prepared and the diluent used, checking for drug incompatibility, and making sure that the dose drawn with a syringe is correct, i.e., matches the drug label. Currently, all the aforesaid details in a preparation process require repeated manual checks to prevent errors in, or failure of, medication preparation.

In addition, a medication preparation operator is required to perform the “three checks, five rights” procedure before adding a drug, and to count the number of empty vials, as well as check if the volume of the prepared liquid is correct, after adding the drug. That is to say, there are stringent regulations concerning the steps of medication preparation, the order of such steps, the doses to be used, and how drugs should be checked against their prescriptions. Since all the details involved have to be manually checked one after another, the higher the complexity of the drug to be prepared, the more the labor and time required for repeated checks.

In view of the above, the present invention provides a system for verifying chemotherapy medication preparation and a method thereof. The system and method carry out verification by image recognition and by sequentially comparing all the details in a preparation process against predetermined information, and can alert the preparation personnel in real time to whether or not the step being performed, the order of the step performed, or the drug being used is correct, thereby greatly reducing errors in the preparation process and increasing the efficiency of medication preparation.

### BRIEF SUMMARY OF THE INVENTION

The primary objective of the present invention is to provide a system for verifying chemotherapy medication preparation. The system uses an image sensing module and an image recognition module to perform image recognition in each step of a preparation process, and a computation and processing module to sequentially verify the identification results and thereby complete verification of the drug prepared.

The second objective of the present invention is to provide a method for verifying chemotherapy medication preparation. The method includes steps for identifying and verifying a preparation action image, a drug image, a syringe marking image, and a mixed-drug image in order to greatly reduce errors in a manually checking process.

To achieve the foregoing objectives, one embodiment of the present invention discloses a system for verifying chemotherapy medication preparation, wherein the system includes: an image sensing module for capturing image information of a chemotherapy medication preparation process; an image recognition module that is in signal connection with the image sensing module and is configured to perform image recognition based on the image information and produce the corresponding identification result; and a computation and processing module that is in signal connection with the image recognition module and is configure to perform computation and comparison based on the identification result; to produce a verification result; to output feedback information when the verification result indicates a mismatch, in order for the image information to be recaptured and re-subjected to image recognition until the verification result indicates a match; and to output preparation process information when the verification result indicates a match.

In a preferred embodiment, the image information includes a preparation action image, a drug image, a syringe marking image, and a mixed-drug image, and the image recognition module produces a preparation action identification result by performing image recognition based on the preparation action image, produces a drug identification result by performing image recognition based on the drug image, produces a dose identification result by performing image recognition based on the syringe marking image, and produces a mixed-drug identification result by performing image recognition based on the mixed-drug image.

In a preferred embodiment, the computation and processing module produces a preparation action comparison result by comparing the preparation action identification result against predetermined preparation action information, produces a drug comparison result by comparing the drug identification result against predetermined drug information, produces a dose comparison result by comparing the dose identification result against predetermined dose information, and produces a mixed-drug comparison result by comparing the mixed-drug identification result against predetermined mixed-drug information.

In a preferred embodiment, the system further includes a database that is in signal connection with the computation and processing module and configured to store the preparation process information.

In a preferred embodiment, the system further includes a display unit that is in signal connection with the computation and processing module and configured to display the image information, the identification result, the verification result, and the feedback information, wherein the display unit is selected from smart glasses.

In a preferred embodiment, the image sensing module captures barcode information of a prescription sheet, the image recognition module performs image recognition based on the barcode information and thereby obtains the corresponding prescription information, and the computation and processing module performs evaluation based on the prescription information and produces an evaluation result. When the evaluation result indicates a match, the image sensing module starts to capture the image information of the chemotherapy medication preparation process. When the evaluation result indicates a mismatch, remark information is output, and the chemotherapy medication preparation process is stopped.

In a preferred embodiment, the system further includes an alert module that is in signal connection with the computation and processing module and configured to output and display alert information when the verification result or the evaluation result indicates a mismatch.

To achieve the second objective, one embodiment of the present invention discloses a method for verifying chemotherapy medication preparation, wherein the method includes the steps of: detecting a preparation action; capturing and identifying a preparation action image corresponding to the preparation action, and producing a preparation action identification result; comparing the preparation action identification result against predetermined preparation action information, and producing a preparation action comparison result; outputting first feedback information when the preparation action comparison result indicates a mismatch, in order to recapture and re-identify the preparation action image until the preparation action comparison result indicates a match, and detecting a drug grabbing action when the preparation action comparison result indicates a match; capturing and identifying a drug image corresponding to the drug grabbing action, and producing a drug identification result; comparing the drug identification result against predetermined drug information, and producing a drug comparison result; outputting second feedback information when the drug comparison result indicates a mismatch, in order to recapture and re-identify the drug image until the drug comparison result indicates a match, and detecting a syringe-based drawing action when the drug comparison result indicates a match; capturing and identifying a syringe marking image corresponding to the syringe-based drawing action, and producing a dose identification result; comparing the dose identification result against predetermined dose information, and producing a dose comparison result; outputting third feedback information when the dose comparison result indicates a mismatch, in order to recapture and re-identify the syringe marking image until the dose comparison result indicates a match, and detecting an action of mixing drugs that are used to form a pharmaceutical preparation, when the dose comparison result indicates a match; capturing and identifying a mixed-drug image corresponding to the action of mixing drugs, and producing a mixed-drug identification result; comparing the mixed-drug identification result against predetermined mixed-drug information, and producing a mixed-drug comparison result; and outputting fourth feedback information when the mixed-drug comparison result indicates a mismatch, in order to recapture and re-identify the mixed-drug image until the mixed-drug comparison result indicates a match, and outputting preparation process information when the mixed-drug comparison result indicates a match.

In a preferred embodiment, the step of detecting a preparation action includes: capturing barcode information of a prescription sheet to obtain the corresponding prescription information; comparing the prescription information against predetermined medication information; producing an evaluation result as to whether or not the prescription information matches the predetermined medication information; and detecting a preparation action when the evaluation result indicates a match; or outputting remark information and suspending the subsequent process of chemotherapy medication preparation, when the evaluation result indicates a mismatch.

In a preferred embodiment, the following steps are performed after the preparation process information is output in response to the mixed-drug comparison result indicating a match: transmitting the preparation process information to a database to store the preparation process information in the database, and outputting notification information to prompt a user to pick up the mixed drug prepared.

The present invention is advantageous in that it can be used to check the process flow of chemotherapy medication preparation effectively, thereby reducing the load on those who are involved in chemotherapy medication preparation.

### BRIEF DESCRIPTION OF THE SEVERAL VIEWS OF THE DRAWINGS

FIG. 1 schematically shows the system according to an embodiment of the present invention;

FIG. 2 shows a flowchart of the method according to another embodiment of the invention;

FIG. 3A schematically shows a step of the method whose flowchart is presented in FIG. 2;

FIG. 3B schematically shows another step of the method whose flowchart is presented in FIG. 2;

FIG. 3C schematically shows still another step of the method whose flowchart is presented in FIG. 2; and

FIG. 3D schematically shows yet another step of the method whose flowchart is presented in FIG. 2.

### DETAILED DESCRIPTION OF THE INVENTION

The effects and features, as well as the foregoing and other objectives, of the present invention can be better understood by referring to the following detailed description of some preferred embodiments.

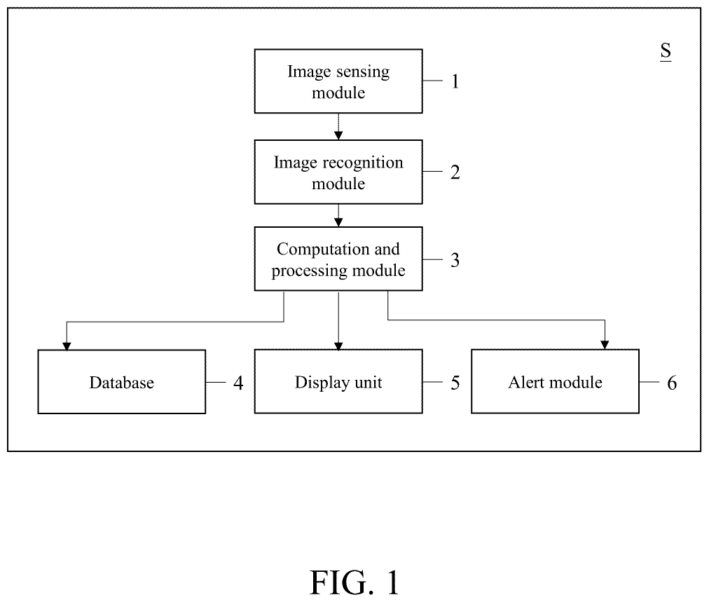

Please refer to FIG. 1, which schematically shows the system according to an embodiment of the present invention. As shown in FIG. 1, the system S for verifying chemotherapy medication preparation according to an embodiment of the invention includes an image sensing module 1, an image recognition module 2, a computation and processing module 3, a database 4, a display unit 5, and an alert module 6. The image recognition module 2 is in signal connection with the image sensing module 1 and the computation and processing module 3 separately. The database 4, the display unit 5, and the alert module 6 are in signal connection with the computation and processing module 3 separately. The way in which each of the foregoing elements of the system S works is detailed as follows.

The image sensing module 1 is configured to capture image information of a chemotherapy medication preparation process. In one embodiment, the image information includes but is not limited to a prescription sheet image, a preparation action image, a drug image, a syringe marking image, and a mixed-drug image.

In one embodiment, a user may put on a pair of smart glasses A mounted with the image sensing module 1 in order to capture the image information of a preparation process with the image sensing module 1. Alternatively, a stand-alone camera device may be used for image sensing. The present invention has no limitation on whether or not the image sensing module 1 is independently provided.

The image recognition module 2 is configured to perform image recognition based on the image information, i.e., to identify the image information, and produce the corresponding identification result. In one embodiment, an image can be captured of each step of a medication preparation process and then subjected to image recognition. The image recognition module 2, however, is not necessarily so designed. Preferably, the image recognition module 2 can carry out image recognition based on a preparation action image and produce the corresponding preparation action identification result, can carry out image recognition based on a drug image and produce the corresponding drug identification result, can carry out image recognition based on a syringe marking image and produce the corresponding dose identification result, and can carry out image recognition based on a mixed-drug image and produce the corresponding mixed-drug identification result. In other words, it is preferable that the image recognition module 2 performs image recognition on each detail in the process flow in order to better inspect each step of the preparation process for defect.

In one embodiment, the image sensing module 1 captures a prescription sheet image, e.g., an image of the QR code on a prescription sheet, and the image recognition module 2 obtains the corresponding prescription information by identifying the QR code.

The computation and processing module 3 is configured to compare the identification result against predetermined information of the corresponding step and produce the corresponding verification result; output feedback information when the verification result indicates a mismatch, in order for the image information to be recaptured and re-subjected to image recognition until the verification result indicates a match, wherein the feedback information may guide or prompt the user to make the correct action; and output preparation process information of the chemotherapy mediation preparation process when the verification result indicates a match. In one embodiment, the computation and processing module 3 can sequentially verify the identification results of the steps identified by the image recognition module 2, but the computation and processing module 3 does not necessarily work in this way. Preferably, the computation and processing module 3 can compare a preparation action identification result against predetermined preparation action information and produce a preparation action comparison result, can compare a drug identification result against predetermined drug information and produce a drug comparison result, can compare a dose identification result against predetermined dose information and produce a dose comparison result, and can compare a mixed-drug identification result against predetermined mixed-drug information and produce a mixed-drug comparison result. In other words, it is preferable that the computation and processing module 3 can compare and check the identification results of different steps and thereby verify the accuracy of each single step.

In one embodiment, the prescription information of a medication preparation process is obtained and evaluated at the beginning of the medication preparation process in order to produce the corresponding evaluation result. The evaluation may involve assessing the reasonability of the prescribed drugs in the prescription information according to the patient's medication history, disease type(s), and so on. If the evaluation result indicates a match, the image sensing module 1 will start capturing the image information of the medication preparation steps that follow. If, however, the evaluation result indicates a mismatch, remark information will be output, and the subsequent process of medication preparation will be suspended. The remark information may be suggested medication information or a notification to the related medical personnel in order for the prescription to be reconfirmed.

The database 4 is configured to store preparation process information. In one embodiment, the database 4 may be a cloud-based storage space or a storage space in a physical server. Once preparation process information is input into the database 4, the database 4 can be used for inventory management, i.e., management of the quantities, expiration dates, and so on of drugs. The database 4 may be further linked to a traceability system (not shown) so that a user can check the flows of drugs at any time.

The display unit 5 is configured to display the image information, the identification result, the verification result, and the feedback information. The display unit 5 may be a pair of smart glasses A that project and thereby display a virtual image, or the display device of other electronic equipment. The display unit 5 is connected to, and configured to communicate with, the computation and processing module 3 through a network. For example, a person responsible for conducting a medication preparation process may wear the smart glasses A while performing the preparation steps so that during the process, other related medical personnel can view the person's preparation operations through the display screen of a computer simultaneously; the display unit 5, however, does not necessarily work in this way.

The alert module 6 is configured to output and display alert information when the verification result or evaluation result indicates a mismatch. In one embodiment, the alert information may be presented in the form of text, graphics, or voice. For example, when the dose comparison result indicates a mismatch, the alert module 6 may produce a text and voice prompt to the effect that the dose is too high or too low and should be measured again; the alert module 6, however, does not necessarily work in this way.

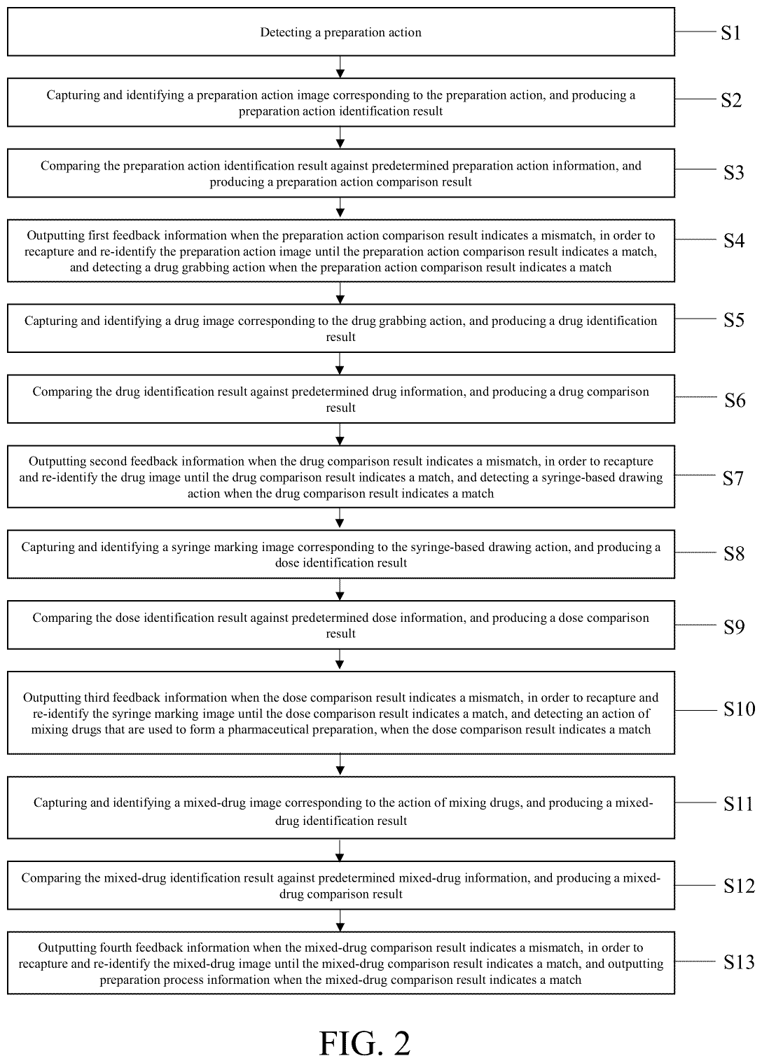

To better explain how the embodiment in FIG. 1 operates, reference is now made to FIG. 2 for a flowchart of the method according to a second embodiment of the present invention. As shown in FIG. 2, the method for verifying chemotherapy medication preparation according to the second embodiment of the invention includes the following steps:
Step S1: detecting a preparation action;
Step S2: capturing and identifying a preparation action image corresponding to the preparation action, and producing a preparation action identification result;
Step S3: comparing the preparation action identification result against predetermined preparation action information, and producing a preparation action comparison result;
Step S4: outputting first feedback information when the preparation action comparison result indicates a mismatch, in order to recapture and re-identify the preparation action image until the preparation action comparison result indicates a match, and detecting a drug grabbing action when the preparation action comparison result indicates a match;
Step S5: capturing and identifying a drug image corresponding to the drug grabbing action, and producing a drug identification result;
Step S6: comparing the drug identification result against predetermined drug information, and producing a drug comparison result;
Step S7: outputting second feedback information when the drug comparison result indicates a mismatch, in order to recapture and re-identify the drug image until the drug comparison result indicates a match, and detecting a syringe-based drawing action when the drug comparison result indicates a match;
Step S8: capturing and identifying a syringe marking image corresponding to the syringe-based drawing action, and producing a dose identification result;
Step S9: comparing the dose identification result against predetermined dose information, and producing a dose comparison result;
Step S10: outputting third feedback information when the dose comparison result indicates a mismatch, in order to recapture and re-identify the syringe marking image until the dose comparison result indicates a match, and detecting an action of mixing drugs that are used to form a pharmaceutical preparation, when the dose comparison result indicates a match;
Step S11: capturing and identifying a mixed-drug image corresponding to the action of mixing drugs, and producing a mixed-drug identification result;
Step S12: comparing the mixed-drug identification result against predetermined mixed-drug information, and producing a mixed-drug comparison result; and
Step S13: outputting fourth feedback information when the mixed-drug comparison result indicates a mismatch, in order to recapture and re-identify the mixed-drug image until the mixed-drug comparison result indicates a match, and outputting preparation process information when the mixed-drug comparison result indicates a match.

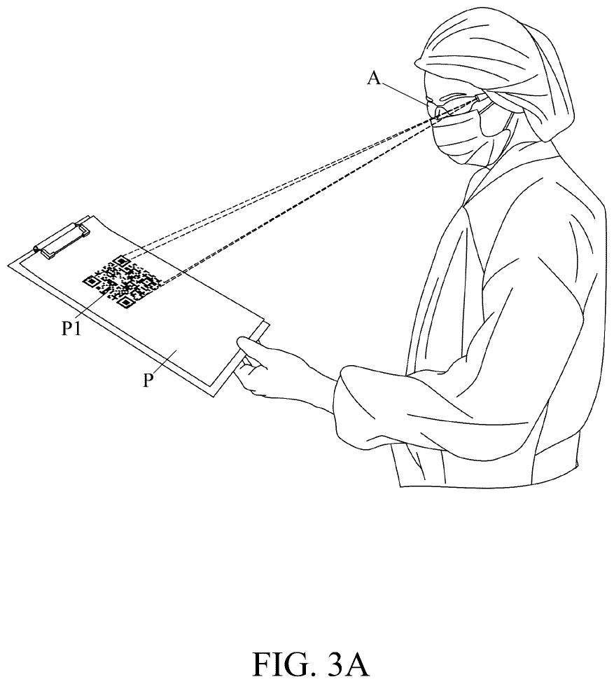

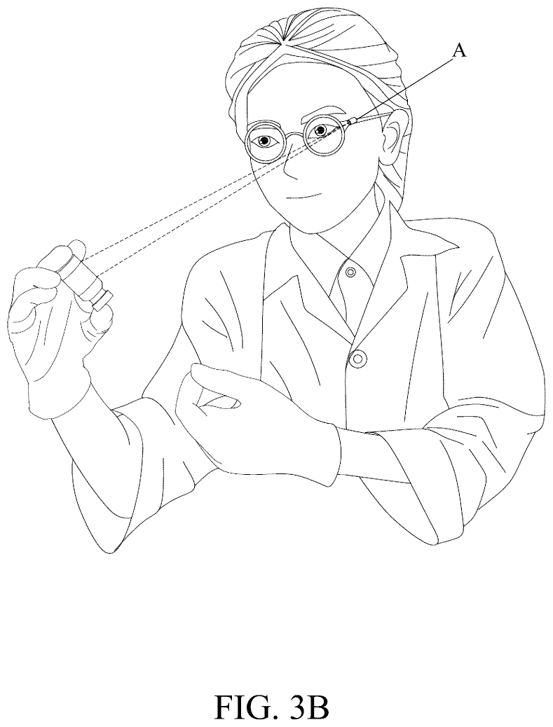

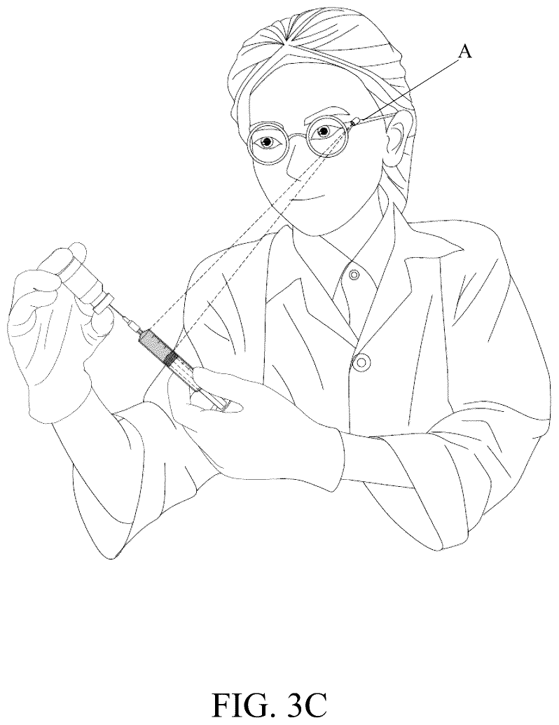

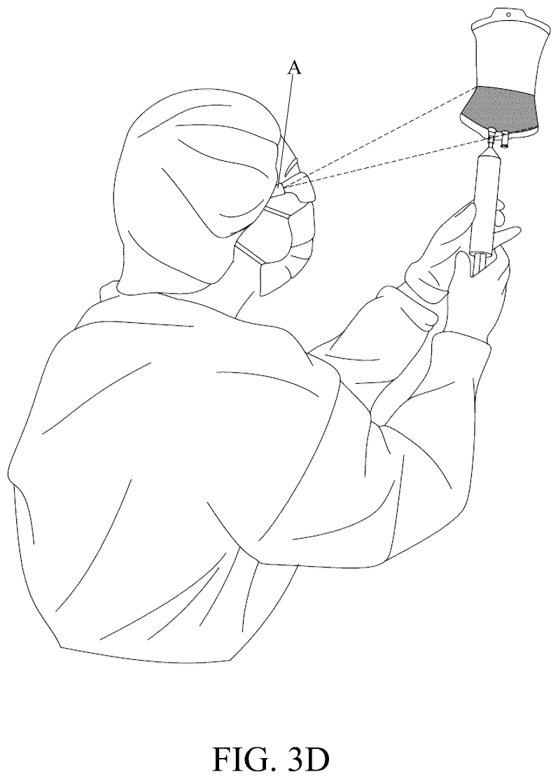

Step S1 can be started as soon as a person responsible for conducting a medication preparation process begins the preparation process, in order to detect the person's action in each step of the preparation process. In one embodiment, referring to FIG. 3A, which schematically shows a step of the method in this embodiment of the present invention, the following are performed at the beginning of a medication preparation process: capturing the barcode information P1 on a prescription sheet P in order to obtain the corresponding prescription information; evaluating whether or not the prescription information matches predetermined medication information; and producing the corresponding evaluation result. More specifically, the barcode, or QR code as shown in FIG. 3A, on the prescription sheet P may link to the prescription information corresponding to the prescription sheet P so that the drug data in the prescription information can be compared against the predetermined medication information. If the evaluation result indicates a match, the subsequent medication preparation process will be carried out. If, however, the evaluation result indicates a mismatch, remark information will be output, and the medication preparation process will be stopped. The remark information may provide the person responsible for conducting the medication preparation process with a suggested medication solution or alert the person that the prescription information has to be checked for errors.

In step S2, a preparation action image corresponding to the preparation action detected is captured and identified in order to produce a preparation action identification result. In one embodiment, the preparation action may include but is not limited to a sterilization step, a drug collecting/gathering step, a drug adding step, and a rechecking step. In fact, all the steps of a medication preparation process may be sequentially identified and have their respective preparation action identification results; step S2, however, is not necessarily so designed.

In step S3, the preparation action identification result is compared against predetermined preparation action information in order to produce the corresponding preparation action comparison result. The predetermined preparation action information can be set according to practical needs.

If the preparation action comparison result indicates a mismatch, first feedback information will be output in step S4. The first feedback information may be an image showing the correct preparation action for reference by the user (i.e., the person conducting the preparation process) or a text or voice message that explains the correct preparation step to the user, so that the user can perform the action again accordingly, with the preparation action image recaptured and re-identified, the objective being to repeat the preparation action and the recapture and re-identification of the preparation action image until the preparation action comparison result indicates a match. When the preparation action comparison result indicates a match, step S4 continues by detecting a drug grabbing action, e.g., the action of grabbing drug A in order to draw drug A. Referring to FIG. 3B, which schematically shows a step of the method in this embodiment of the present invention, the detection target may be a characteristic of the drug being grabbed, of the bottle containing the drug, of the box containing the drug, or of the bag containing the drug. It is worth reiteration that when the preparation action comparison result indicates a mismatch, the person conducting the preparation process will be alerted by the first feedback information that the step is erroneously performed and/or be reminded by the first feedback information of the step to be performed. Only when the preparation action comparison result indicates a match will the next step begin.

In step S5, a drug image corresponding to the drug grabbing action is captured and identified, and by doing so, the drug being grabbed can be identified according to a characteristic of the drug, of the bottle containing the drug, of the box containing the drug, or of the bag containing the drug. A drug identification result is then produced.

In step S6, the drug identification result is compared against predetermined drug information in order to produce a drug comparison result. For example, if step A requires drug A to be grabbed first, the drug comparison result will indicate a match when the identification result is drug A, and a mismatch when the identification result is drug B. The predetermined drug information, therefore, can be set according to practical needs.

If the drug comparison result indicates a mismatch, second feedback information will be output in step S7. The second feedback information may be an image showing the correct drug image for reference by the user or a text or voice message either alerting the user that the wrong drug is grabbed or reminding the user of the drug to be grabbed, so that the user can grab the correct drug accordingly, with the drug image recaptured and re-identified, the objective being to repeat the drug grabbing action and the recapture and re-identification of the drug image until the drug comparison result indicates a match. When the drug comparison result indicates a match, step S7 continues by detecting a syringe-based drawing action.

In step S8, a syringe marking image corresponding to the syringe-based drawing action is captured and identified in order to produce a dose identification result. For example, referring to FIG. 3C, which schematically shows a step of the method in this embodiment of the present invention, the person conducting the preparation process starts a syringe-based drawing action, and when the drawing action stops, the corresponding syringe marking image is identified.

In step S9, the dose identification result is compared against predetermined dose information in order to produce a dose comparison result. In one embodiment, the predetermined dose information can be adjusted according to each drug to be used, e.g., 5 c.c. of drug A, 3 c.c. of drug B, and 1 c.c. of drug C, so that with the previous steps determining each drug that is currently grabbed, this step further determines whether or not the dose corresponding to the drug currently grabbed has been drawn. The foregoing examples of drugs A, B, and C and their respective doses are not limiting.

If the dose comparison result indicates a mismatch, third feedback information will be output in step S10. The third feedback information may be an image showing the current syringe marking image and the correct syringe marking image, or a text or voice message alerting the user that the dose is too high or too low, so that the user can perform the syringe-based drawing action again accordingly, with the syringe marking image recaptured and re-identified, the objective being to repeat the syringe-based drawing action and the recapture and re-identification of the syringe marking image until the dose comparison result indicates a match. When the dose comparison result indicates a match, step S10 continues by detecting an action of mixing drugs that are used to form a pharmaceutical preparation.

In step S11, a mixed-drug image corresponding to the action of mixing drugs is captured and identified in order to produce a mixed-drug identification result. In one embodiment, referring to FIG. 3D, which schematically shows a step of the method in this embodiment of the present invention, it is required to check the color, mixing uniformity, and total quantity of the mixed drug and whether or not the mixed drug contains any foreign matter or impurity. By identifying the mixed-drug image, it can be determined whether or not a mistake has been made in the preparation process. The foregoing items to be checked, however, are not limiting.

In step S12, the mixed-drug identification result is compared against predetermined mixed-drug information in order to produce a mixed-drug comparison result. As mentioned in relation to the previous step, the predetermined mixed-drug information may include the intended color, mixing uniformity, total quantity, foreign matter/impurity content, and so on of each of a plurality of mixed pharmaceutical preparations prepared from different drugs, so as to reconfirm whether or not the mixed pharmaceutical preparations meet their respective requirements.

If the mixed-drug comparison result indicates a mismatch, fourth feedback information will be output in step S13. The fourth feedback information may be an image showing the correct mixed-drug image, or a text or voice message indicating which of the current mixed color, mixing uniformity, total quantity, and foreign matter/impurity content is/are incorrect and what are the correct mixed color, mixing uniformity, total quantity, and foreign matter/impurity content, so that the user can re-perform the action of mixing drugs accordingly, with the mixed-drug image recaptured and re-identified, the objective being to repeat the action of mixing drugs and the recapture and re-identification of the mixed-drug image until the mixed-drug comparison result indicates a match. When the mixed-drug comparison result indicates a match, step S13 continues by outputting preparation process information, wherein the preparation process information includes the process flow, contents, and time of the preparation process and the expiration date of the drug prepared, among other related information. The preparation process information may be further transmitted to the database 4 for storage, and corresponding notification information may be output to prompt the user to pick up the mixed drug prepared.

The way in which the foregoing embodiment works is described in more detail below.

After the user puts on the smart glasses A and enters the chemotherapy medication preparation room, the method begins by capturing the QR code on the prescription sheet P in order to obtain the corresponding prescription information and compare the prescription information against predetermined medication information to see whether or not they match. When it is determined that the prescription information matches the predetermined medication information, the user proceeds to perform the preparation action. Each preparation action will be subjected to image recognition and comparison, and only when the current preparation action meets the corresponding predetermined requirement will the next action be allowed to be performed. When the preparation actions involve mixing drugs, drug image identification and comparison will be carried out, and once the drugs are confirmed, syringe marking image identification and comparison will be conducted after each drug is drawn, in order to determine whether or not the dose of each drug meets the corresponding requirement. After the drugs are mixed, mixed-drug image identification and comparison will be performed to make sure that the color, total quantity, uniformity, and foreign matter/impurity content of the mixed drug prepared match their respective descriptions in the prescription information. When the final mixed-drug comparison result indicates a match, the preparation process information of this chemotherapy medication will be output. The preparation process information may be stored in the database or transmitted to a traceability system so that those who intend to use this drug can track the flow of the drug at any time.

During the process stated above, each identification result and the corresponding comparison result may be sequentially displayed by the smart glasses A as an identification result image and a comparison result image, or when the comparison result corresponding to an identification result indicates a mismatch, the smart glasses A may display an alert image and thereby prompt the user to re-perform the action just taken, make an adjustment, stop the process, or follow other instructions. In either case, any image captured by the smart glasses A and the corresponding identification result and comparison result may be simultaneously displayed by an electronic device at an arbitrary location other than the chemotherapy medication preparation room in order to be viewed and monitored by other people, and when an identification result is ambiguous or a comparison result indicates a mismatch, the people monitoring the process from afar will see the alert image at the same time and may send a text message, voice message, and/or image through the electronic device to the smart glasses A in order to communicate with the glass wearer. In other words, other related personnel can help double-check the medication preparation process in order to ensure that the process is correctly performed, and there is no limitation on the location of the monitoring personnel. Thus, the present invention uses image recognition to not only contribute to the correct operation of a chemotherapy medication preparation process, but also shorten the time required for doing repeated manual checks during the preparation process.

In one embodiment, the comparison results, identification results, and other data stored in the database can be calibrated by artificial intelligence, with text, voice, or image feedback introduced into the calibration process so that comparison results can be modified in real time according to the properties of the drugs in use or the preparation method in use, thereby enhancing the accuracy of identification and comparison.

According to the above, the present invention provides a system for verifying chemotherapy medication preparation and a method thereof, wherein all the images captured by the image sensing module during a chemotherapy medication preparation process are sequentially identified by the image recognition module and then compared by the computation and processing module for further verification. Thus, accurate rechecks as well as an increase in the preparation efficiency of chemotherapy drugs can be achieved to attain the objectives of the invention.

It should be pointed out that the embodiments described above are only some preferred ones of the present invention and are not intended to be restrictive of the scope of the invention. Any simple equivalent change or modification that is made according to the appended claims and the contents disclosed herein shall fall within the scope of the invention.

What is claimed is:

1. A system for verifying chemotherapy medication preparation, comprising:
an image sensing module for capturing image information of a chemotherapy medication preparation process;
an image recognition module in signal connection with the image sensing module, wherein the image recognition module performs image recognition based on the image information and produces a corresponding identification result; and
a computation and processing module in signal connection with the image recognition module, wherein the computation and processing module performs computation and comparison based on the identification result; produces a verification result; outputs feedback information when the verification result indicates a mismatch, in order for the image information to be recaptured and re-subjected to the image recognition until the verification result indicates a match; and outputs preparation process information when the verification result indicates a match.

2. The system for verifying chemotherapy medication preparation as claimed in claim 1, wherein the image information comprises a preparation action image, a drug image, a syringe marking image, and a mixed-drug image, and the image recognition module performs the image recognition based on the preparation action image and produces a preparation action identification result, performs the image recognition based on the drug image and produces a drug identification result, performs the image recognition based on the syringe marking image and produces a dose identification result, and performs the image recognition based on the mixed-drug image and produces a mixed-drug identification result.

3. The system for verifying chemotherapy medication preparation as claimed in claim 2, wherein the computation and processing module compares the preparation action identification result against predetermined preparation action information and produces a preparation action comparison result, compares the drug identification result against predetermined drug information and produces a drug comparison result, compares the dose identification result against predetermined dose information and produces a dose comparison result, and compares the mixed-drug identification result against predetermined mixed-drug information and produces a mixed-drug comparison result.

4. The system for verifying chemotherapy medication preparation as claimed in claim 1, further comprising a database in signal connection with the computation and processing module, wherein the database stores the preparation process information.

5. The system for verifying chemotherapy medication preparation as claimed in claim 1, further comprising a display unit in signal connection with the computation and processing module, wherein the display unit displays the image information, the identification result, the verification result, and the feedback information, and the display unit is selected from smart glasses.

6. The system for verifying chemotherapy medication preparation as claimed in claim 1, wherein the image sensing module captures barcode information of a prescription sheet, the image recognition module performs the image recognition based on the barcode information and thereby obtains corresponding prescription information, and the computation and processing module performs evaluation based on the prescription information and produces an evaluation result, wherein when the evaluation result indicates a match, the image sensing module starts to capture the image information of the chemotherapy medication preparation process, and when the evaluation result indicates a mismatch, remark information is output, and the chemotherapy medication preparation process is stopped.

7. The system for verifying chemotherapy medication preparation as claimed in claim 6, further comprising an alert module in signal connection with the computation and processing module, wherein the alert module outputs and displays alert information when the verification result or the evaluation result indicates a mismatch.

8. A method for verifying chemotherapy medication preparation, comprising the steps of:
detecting a preparation action;
capturing and identifying a preparation action image corresponding to the preparation action, and producing a preparation action identification result;
comparing the preparation action identification result against predetermined preparation action information, and producing a preparation action comparison result;
outputting first feedback information when the preparation action comparison result indicates a mismatch, in order to recapture and re-identify the preparation action image until the preparation action comparison result indicates a match, and detecting a drug grabbing action when the preparation action comparison result indicates a match;
capturing and identifying a drug image corresponding to the drug grabbing action, and producing a drug identification result;
comparing the drug identification result against predetermined drug information, and producing a drug comparison result;
outputting second feedback information when the drug comparison result indicates a mismatch, in order to recapture and re-identify the drug image until the drug comparison result indicates a match, and detecting a syringe-based drawing action when the drug comparison result indicates a match;
capturing and identifying a syringe marking image corresponding to the syringe-based drawing action, and producing a dose identification result;
comparing the dose identification result against predetermined dose information, and producing a dose comparison result;
outputting third feedback information when the dose comparison result indicates a mismatch, in order to recapture and re-identify the syringe marking image until the dose comparison result indicates a match, and detecting an action of mixing drugs used to form a pharmaceutical preparation, when the dose comparison result indicates a match;
capturing and identifying a mixed-drug image corresponding to the action of mixing drugs, and producing a mixed-drug identification result;
comparing the mixed-drug identification result against predetermined mixed-drug information, and producing a mixed-drug comparison result; and
outputting fourth feedback information when the mixed-drug comparison result indicates a mismatch, in order to recapture and re-identify the mixed-drug image until the mixed-drug comparison result indicates a match, and outputting preparation process information when the mixed-drug comparison result indicates a match.

9. The method for verifying chemotherapy medication preparation as claimed in claim 8, wherein the step of detecting a preparation action comprises: capturing barcode information of a prescription sheet to obtain corresponding prescription information; comparing the prescription information against predetermined medication information; producing an evaluation result as to whether or not the prescription information matches the predetermined medication information; and detecting a preparation action when the evaluation result indicates a match; or outputting remark information and suspending a subsequent process of the chemotherapy medication preparation, when the evaluation result indicates a mismatch.

10. The method for verifying chemotherapy medication preparation as claimed in claim 8, wherein after the preparation process information is output in response to the mixed-drug comparison result indicating a match, the preparation process information is transmitted to and stored in a database, and notification information is output to prompt a user to pick up the mixed drug prepared.
